(19) United States
(12) Patent Application Publication
(10) Pub. No.: US 2003/0106127 A1
(43) Pub. Date: Jun. 12, 2003

(54) Visor and method for exhibiting a display thereon

(76) Inventors: Kenneth A. Shwartz, Mattapoisett, MA (US); Charles Lord, Mattapoisett, MA (US)

Correspondence Address: FOLEY HOAG, LLP, PATENT GROUP, WORLD TRADE CENTER WEST, 155 SEAPORT BLVD, BOSTON, MA, 02110, US

(21) Appl. No.: 10/020,127

(22) Filed: Dec. 7, 2001

(51) Int. Cl. A61F 9/00

(52) U.S. Cl. 2/12; 40/329

(57) ABSTRACT

A headgear and method for exhibiting displays, such as company logos, special events, messages, or just general advertisement on the bill of the headgear are provided. The bill can be made from a flexible material sufficiently pliable to permit a display to be embroidered or sewn therethrough, while permitting the bill to maintain its shape. The headgear further includes a resilient headband for engagement against the sides of the head of a user.

### TECHNICAL FIELD

The present invention relates generally to headgear, for examples visors, and more particularly to headgear having a bill which permits exhibition of displays thereon.

### BACKGROUND ART

Headgear, including visors and caps, have been in use for quite some time to display company logos, special events, messages, or just general advertisement. The exhibition of these displays have mostly been on the front of the visor or cap, and in particular, in the area of the visor or cap which is adjacent the forehead of the wearer, where the fabric is sufficiently soft to permit, for instance, embroidering or sewing of the display. To a certain extent, some of the displays have been provided on the bill of the visor or cap. Such displays, however, have mostly been embroidered or sewn on to the soft fabric covering the bill and not to the bill itself. One reason which may have prevented the display from being sewn or embroidered directly through the bill may be because most bills, being made from hard plastic or cardboard, are too stiff to permit sewing or embroidering of displays. Many commercially available visors and caps are designed to include a bill that is sufficiently stiff to permit the bill to maintain its shape when the visor or cap is worn. Such a bill may be adequate for many purposes and may be desirable in many situations. However, a stiff bill typically does not permit a display to be easily attached or embroidered therethrough.

The exhibition of displays on the bill of the headgear may also be an expensive procedure. In particular, as the fabric around the stiff bill has already been stretched tightly over the bill, manufactures cannot use the existing headgear in their warehouses, as is, to embroider the display onto the fabric around the bill. Instead, in one practice, the entire bill or the fabric over the entire bill must be removed, the display embroidered on to a new piece of fabric, and subsequently the bill be re-covered with the newly embroidered fabric. This process can be avoided if the headgear manufacturers is given sufficient notice. With notice, the manufacturers can modify the manufacturing process to accommodate the embroidering of the display onto the piece of fabric prior to covering the bill. Such modification of the process, however, may require the fabric around the bill to be set aside for the embroidering, which again can be time consuming and may add unwanted expenses to the production of the headgear. As is more often than not, the amount of the order may not justify modification of the normal headgear manufacturing process. Accordingly, for those organizations wishing to have their logos, messages, or special events exhibited on the bill, unless the they can foresee a sufficient return on the sales of the headgear with such displays, many will forego the exhibition of the display on the bill of the headgear.

### SUMMARY OF THE INVENTION

The present invention, in accordance with one embodiment, provides a headgear, for example, a visor, which can exhibit displays, such as company logos, special events, messages, or just general advertisement. The headgear, as provided, can include a headband portion for placement circumferentially about a head of a person. The headgear can also include a bill portion having distal end and a proximal end, which proximal end is attached to the headband portion. The bill portion, in an embodiment of the invention, may be made from a material that is sufficiently flexible and pliable to permit a display to be embroidered or sewn therethrough, while permitting the bill to maintain its shape. The headgear may also include a resilient headband portion having opposing ends for engaging and fitting around a portion of the head of a user. As the head size may vary from person to person, the opposing ends of the resilient band portion are designed to spread apart when engaging the head of the user, so as to accommodate different size heads.

In accordance with another embodiment of the present invention, a method of exhibiting a display on a headgear is provided. The method may include providing a headgear having a headband portion and a bill portion that is attached at its proximal end to the headband. The bill portion, in one embodiment, may be made from a material that is sufficiently flexible and pliable to permit a display to be embroidered or sewn therethrough, while permitting the bill to maintain its shape. Subsequently, a display may be secured through the flexible material on to the bill portion of the headgear either by embroidering or sewing. Alternatively, the bill portion of the headgear may initially be provided, to which the display may be embroidered or sewn therethrough. Thereafter, the bill portion may be attached at its proximal end to the band.

### BRIEF DESCRIPTION OF THE DRAWINGS

FIG. 1 illustrates a perspective upper frontal view a headgear in accordance with one embodiment of the present invention.

FIG. 2 illustrates a perspective upper rear view of the headgear in FIG. 1.

FIGS. 3-4 illustrate various embodiments for securely exhibiting a display on to the headgear of the present invention.

### DETAILED DESCRIPTION OF SPECIFIC EMBODIMENTS

The present invention provides, in one embodiment, a headgear which permits embroidering or sewing of displays, such as company logos, special events, messages, or just general advertisement, thereon. The headgear of the present invention may either be a visor or a cap.

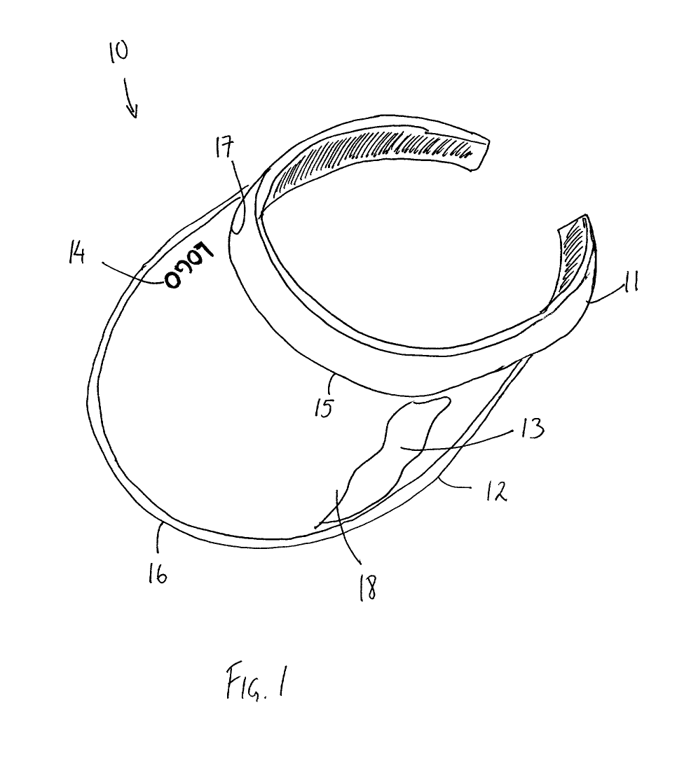

In FIG. 1 there is shown a visor 10 having a bill portion 12 attached to a headband portion 11. The bill portion 12, in one embodiment, comprises a member 13 made from a sufficiently flexible material. The provision of a flexible member 13 in the bill portion 12 permits the bill portion 12 to be sufficiently pliable to allow a display 14 to be embroidered or sewn therethrough, while still permitting the bill portion 12 to adequately maintain its shape. The bill portion 12, as shown in FIG. 1, includes a proximal end 15 and a distal end 16. The proximal end 15 may be designed to have a curvature 17 which complements the curvature about a periphery of the headband portion 11, so that the bill portion 12 may be closely attached at its proximal end 15 to the headband portion 11. It should be appreciated that the curvature 17 may vary according to the size of the headband portion 11, so long as the curvature 17 is maintained in a manner which permits a complementary fit about a periphery of the headband portion 11.

The flexible member 13 comprising the bill portion 12, in one embodiment, is preferably overlaid with a piece of covering 18. To provide the covering 18 with a secure fit over the flexible member 13, the covering 18 may be sewn on to the flexible member 13. Alternatively, the covering 18 may be tightly stretched over the flexible material 13, and the covering 18 and flexible member 13 attached to the headband portion 11. In an embodiment, the material from which the covering 18 may be made includes cotton fabric, polyester fabric, plastic, or other commercially available material typically used in the manufacturing of headgear or clothes.

In order to provide the bill portion 12 with the ability to firmly hold its shape, while still permitting a display 14 to be embroidered or sewn therethrough, in one embodiment of the invention, the flexible member 13 may be provided with a density of at least about 13 pounds per cubic foot (pcf) and preferably in a range of from 13 pcf to about 16.5 pcf. In an embodiment, the flexible member 13 may have a density of from about 16.0 pcf to about 16.5 pcf. Materials which can exhibit these characteristics may be made generally from closed cell foam, for example, neoprene or polyolefin resin substance.

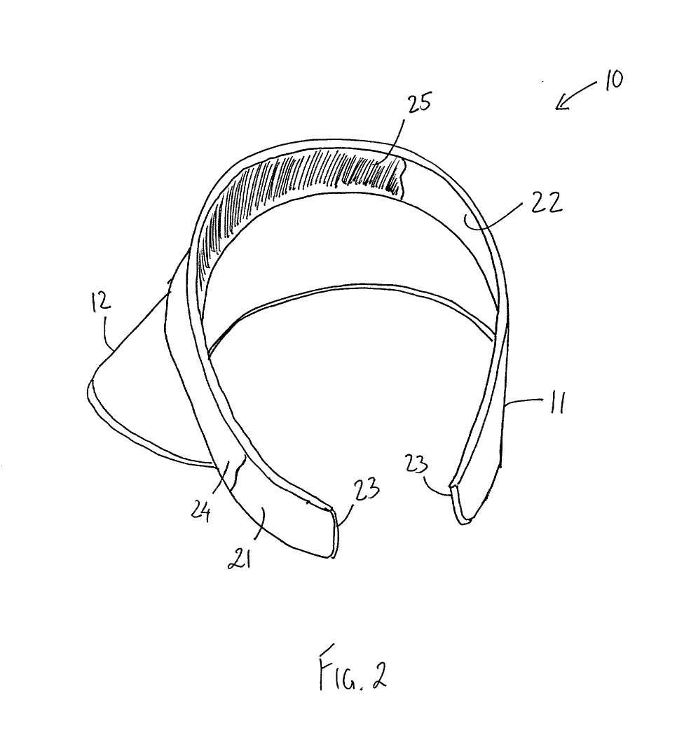

Referring now to FIG. 2, the visor 10 further includes headband portion 11 designed for engagement about a head of a user (not shown). The headband portion 11 includes an outer surface 21 to which the bill portion 12 may be attached, and an inner surface 22 opposite the outer surface 21. In one embodiment, the headband portion 11 may be made from a hard resilient material, such as plastic, wood, or a shape-memory metal or metal alloy, and may include opposing ends 23 spaced apart to permit engagement against the sides of the head of the user. By providing the headband portion 11 with opposing ends 23, different size heads may be accommodated when the visor 10 is in use. In particular, since the headband portion 11 is resilient, the opposing ends 23 may be spread apart from their normal position when the headband portion 11 engages the head of the user. Once on the head of the user, the resiliency of the headband portion 11 urges the opposing ends 23 towards one another, so that the ends 23 will tend to return to their normal position to securely engage the sides of the user's head.

To provide comfort to the user, the headband portion 11 may be overlaid with a piece of covering 24, so that the material from which the headband portion 11 is made does not rest directly against the head of the user. If desired, the covering 24 may be made from the same material and color as that provided for covering 18 in FIG. 1. In one embodiment, a sweatband 25 may also be provided along the inner surface 22 of the headband portion 11. The provision of the sweatband 25 can help to absorb perspiration generated by the user and reduce the amount of perspiration running down the forehead and/or sides of the user's head. The sweatband 25 may be attached to the covering 24 or the inner surface 22 of the headband portion 11 by any conventional means, including stitchings, glue, hook and loop mechanism, or other similar fasteners.

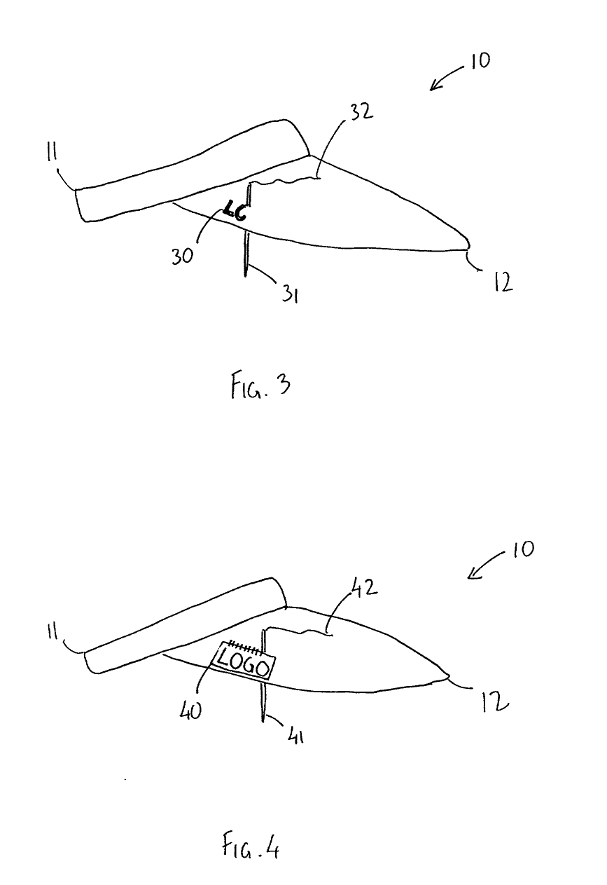

The exhibition of a display on the bill portion 12 of the headgear 10 will now be discussed in accordance with one embodiment of the present invention. To exhibit a display 30 by embroidering, such as that shown in FIG. 3, a location to which the display 30 will be exhibited may first be determined. Subsequently, the bill portion 12, and a machine (not shown) for embroidering the display 30 may be engaged at the location for exhibiting the display 30. The machine may thereafter be initiated to permit embroidering needle 31 to penetrate through the bill portion 12 along with threading 32. The machine may be permitted to continue until the desired display 30 is completed.

To exhibit display 40, for example, by sewing, the display 40, as shown in FIG. 4, may initially be placed directly on the bill portion 12 at a location to which the display 40 will be exhibited. Subsequently, engagement between the display 40, as it is positioned on the bill portion 12, and a machine (not shown), such as a sewing machine, for securely attaching the display 40 to the bill portion 12 may be initiated. The machine may thereafter be activated to permit sewing needle 41 to penetrate through the bill portion 12 along with threading 42. The machine is permitted to continue until the display 40 is securely attached through the bill portion 12.

It should be appreciated that the processes provided in connection with FIGS. 3 and 4 may be modified, so that the securing of the displays 30 or 40 may be accomplished during manufacturing process of the headgear 10. Specifically, the embroidering or sewing of the display 30 or 40 through the bill portion 12 may be accomplished prior to the attachment of the bill portion 12, at its proximal end, to the headband portion 11 of the headgear 10. In this manner, the present invention also contemplates that a plurality of bill portions 12, each exhibiting a different display 30 or 40, may be provided for a headgear designed with a removable bill portion 12, such that depending on the mood of the user or the occasion encountered, the bill portion 12 with one display may be interchanged with another bill portion 12 having another display, so as to exhibit different displays that are appropriate for the specific occasions. The headgear having a removable bill may include any means known in the art, for example, hook and loop fasteners, for removable attachment of the bill to the headgear.

While the invention has been described in connection with the specific embodiments thereof, it will be understood that it is capable of further modification. In particular, although reference has been made to a visor, the flexible bill portion may be modified for use with a cap or other headgears. Furthermore, this application is intended to cover any variations, uses, or adaptations of the invention, including such departures from the present disclosure as come within known or customary practice in the art to which the invention pertains, and as fall within the scope of the appended claims.

What is claimed is:

1. A headgear for exhibiting a display, the headgear comprising:
a resilient headband portion having opposing ends for engagement against sides of a head of a user; and
a bill portion being attached at its proximal end to the headband portion, the bill portion being made from a material that is sufficiently flexible and pliable to permit a display to be embroidered or sewn therethrough, while permitting the bill to maintain its shape.

2. A headgear as set forth in claim 1, wherein the opposing ends of the headband portion are designed to spread apart, so as to accommodate different size heads.

3. A headgear as set forth in claim 1, wherein the opposing ends of the headband portion are designed such that the opposing ends are urged toward one another to securely engage against the sides of the head of a user.

4. A headgear as set forth in claim 1, wherein the material from which the bill portion is made has a density of at least about 13 pcf.

5. A headgear as set forth in claim 1, wherein the material from which the bill portion is made has a density range of from about 13 pcf to about 16.5 pcf.

6. A headgear as set forth in claim 1, wherein the material from which the bill portion is made is closed cell foam.

7. A headgear as set forth in claim 5, wherein the closed cell foam is one of neoprene and polyolefin resin material.

8. A headgear as set forth in claim 1, further including sweatband positioned against a surface of the headband portion opposite the surface to which the bill portion is attached.

9. A method for exhibiting a display on a headgear, the method comprising:
providing a headgear having a resilient headband portion with opposing ends, and a bill portion being attached at its proximal end to the headband, the bill portion being made from material sufficiently flexible and pliable to permit a display to be embroidered or sewn therethrough, while permitting the bill portion to maintain its shape; and
securing through the flexible material a display on the bill portion of the headgear.

10. A method as set forth in claim 9, wherein, in the step of providing, the material from which the bill portion is made includes a density of at least about 13 pcf.

11. A method as set forth in claim 9, wherein the step of securing includes embroidering the display.

12. A method as set forth in claim 9, wherein the step of securing includes:
placing a display directly on to the bill portion of the headgear; and
sewing the display through the flexible material on to the bill portion of the headgear.

13. A method of exhibiting a display on a headgear, the method comprising:
providing a bill portion of a headgear, the bill portion being made from a material sufficiently flexible and pliable to permit a display to be embroidered or sewn therethrough, while permitting the bill portion to maintain its shape;
securing through the flexible material a display on to the bill portion of the headgear;
providing a headband portion for placement against sides of a head of a person; and
attaching the bill portion at its proximal end to the headband portion.

14. A method as set forth in claim 13, wherein, in the step of providing, the material from which the bill portion is made includes a density of at least about 13 pcf.

15. A method as set forth in claim 13, wherein the step of securing includes embroidering the display.

16. A method as set forth in claim 13, wherein the step of securing includes:
placing a display directly on to the bill portion of the headgear; and
sewing the display through the flexible material on to the bill portion of the headgear.
